(19) United States
(12) Patent Application Publication
(10) Pub. No.: US 2006/0020204 A1
(43) Pub. Date: Jan. 26, 2006

(54) System and method for three-dimensional space management and visualization of ultrasound data ("SonoDEX")

(75) Inventors: Luis Serra, Singapore (SG); Chua Beng Choon, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging, S.p.A., Milano (IT)

(21) Appl. No.: 11/172,729

(22) Filed: Jul. 1, 2005

Related U.S. Application Data
(60) Provisional application No. 60/585,214, filed on Jul. 1, 2004.
(60) Provisional application No. 60/660,858, filed on Mar. 11, 2005.
(60) Provisional application No. 60/585,462, filed on Jul. 1, 2004.

(51) Int. Cl. A61B 8/00

(52) U.S. Cl. 600/437

(57) ABSTRACT

A system and method for the imaging management of a 3D space where various substantially real-time scan images have been acquired is presented. In exemplary embodiments according to the present invention, a user can visualize images of a portion of a body or object obtained from a substantially real-time scanner not just as 2D images, but as positionally and orientationally located slices within a particular 3D space. In such exemplary embodiments a user can convert such slices into volumes whenever needed, and can process the images or volumes using known image processing and/or volume rendering techniques. Alternatively, a user can acquire ultrasound images in 3D using the techniques of UltraSonar or 4D Ultrasound. In exemplary embodiments according to the present invention, a user can manage various substantially real-time images obtained, either as slices or volumes, and can control their visualization, processing and display, as well as their registration and fusion with other images, volumes and virtual objects obtained or derived from prior scans of the body or object of interest using various modalities.

### CROSS REFERENCE TO RELATED APPLICATIONS

This application claims the benefit of the following U.S. Provisional Patent Applications: (i) Ser. No. 60/585,214, entitled “SYSTEM AND METHOD FOR SCANNING AND IMAGING MANAGEMENT WITHIN A 3D SPACE (“SonoDEX”)”, filed on Jul. 1, 2004; (ii) Ser. No. 60/585,462, entitled “SYSTEM AND METHOD FOR A VIRTUAL INTERFACE FOR ULTRASOUND SCANNERS (“Virtual Interface”)”, filed on Jul. 1, 2004; and (iii) Ser. No. 60/660,858, entitled “SONODEX: 3D SPACE MANAGEMENT AND VISUALIZATION OF ULTRASOUND DATA”, filed on Mar. 11, 2005.

The following related United States patent applications, under common assignment herewith, are also fully incorporated herein by this reference: Ser. No. 10/469,294 (hereinafter “A Display Apparatus”), filed on Aug. 29, 2003; Ser. No. 10/725,773 (hereinafter “Zoom Slider”), Ser. No. 10/727,344 (hereinafter “Zoom Context”), and Ser. No. 10/725,772 (hereinafter “3D Matching”), each filed on Dec. 1, 2003; Ser. No. 10/744,869 (hereinafter “UltraSonar”), filed on Dec. 22, 2003, and Ser. No. 60/660,563 entitled “A METHOD FOR CREATING 4D IMAGES USING MULTIPLE 2D IMAGES ACQUIRED IN REAL-TIME (“4D Ultrasound”), filed on Mar. 9, 2005.

### TECHNICAL FIELD

The present invention relates to substantially real-time imaging modalities, such as ultrasound or the equivalent, and more precisely relates to the interactive display and manipulation of a three-dimensional space for which a plurality of scans have been performed.

### BACKGROUND OF THE INVENTION

A substantially real-time image produced by a probe, such as, for example, an ultrasound probe, represents a cut through an organ or other 3D anatomical structure of a given patient. Such an image has a 3D position and orientation relative to the patient's depicted organ or other anatomical structure, and knowing this 3D position and orientation is often key to a proper interpretation of the ultrasound image for both diagnostic as well as interventional purposes. As an example of the latter is when, for example, a clinician plans an intervention and must decide precisely where to insert a needle or therapeutically direct an ultrasound beam.

Moreover, key in interpreting substantially real-time images is the time at which a particular image was acquired relative to the time when the scan started. This is especially true in cases where one or more contrast media have been injected into the arteries (or other vessels) of a patient, given the fact that a contrast fluid's signal varies with time as well as organ intake. The body is not a stationary object, but a time-varying one. There is much evidence that indicates that it is not enough to simply observe an organ (or a pathology) as a stationary object but it is necessary to perceive it as part of a time-varying process in order to truly understand its function. The most obvious is the heart, since it moves. One 3D image of gives one view, but to understand the ejection fraction, or to analyze the condition of a valve it is key to visualize its movement. In the case of a tumor, and when using contrast media and ultrasound, what happens is that the contrast flows through the arteries, then reaches and fills the tumor, and then washes out. It is important to visualize the entire process (wash in and wash out) to understand how vessels are feeding the tumor, as well as how much blood is the tumor taking in, in order to understand its aggressiveness. There is no single picture that can show this process. One at best can capture the image (or volume) that shows the time point when the contrast is filling the tumor at its maximum, but that misses the time when the vessels are visible. Thus, the rate of contrast intake is important in order to diagnose and understand the pathology.

Moreover, having a volume (and not just a slice with position and orientation) is essential to any quantification process. If there is only a probe cutting through an organ that is moving (due, for example, to breathing or due to its own movement, such as, for example, the heart) the resulting image can be hard to compare against another image taken a fraction of a second later since the organ in question will have moved and thus the cut will be in another, slightly shifted, part of the organ. However, if a comparison is made from one volume to another volume, such error can be minimized since the volume is made of several cuts and it averages the positioning problem.

Notwithstanding the interpretational value of such additional information, historically conventional ultrasound scanners, for example, simply displayed a ‘flat’ image of the cutting plane into a given organ of interest, and provided no reference as to the relative position of the displayed cutting plane relative to anatomical context or to the displayed cut's acquisition time.

To remedy this problem, state of the art ultrasound scanners, such as, for example, models manufactured by Kretz (now a GE company) and Philips, added 3D volumetric acquisition capabilities to their ultrasound probes. As a result they can display a 4D volume (i.e., a volume that changes with time) by producing a series of acquired images that can then be reconstructed into a volume. The resulting volume can then be displayed (after appropriate resampling) using standard volume rendering techniques. Nonetheless, while the individual slices comprising such a volume are loosely registered to each other (loosely because the subject's body is moving throughout the acquisition, and thus the body does not have a fixed spatial relationship to the probe during the acquisition) they are not registered in any sense to the 3D patient space.

Moreover, even if such a volume is acquired and displayed, the physical interfaces provided to manipulate these volumes are not themselves three-dimensional, generally being nothing more than a standard computer keyboard and mouse (or the equivalent, such as a trackball). Accordingly, using such tools to effect 3D operations necessitates awkward mappings of 3D manipulations onto essentially 2D devices. The necessity of such awkward mappings may be one of the reasons why 3D visualization has not gained the acceptance in the medical community that it may be due.

Additionally, some systems, such as, for example, the Esaote™ virtual navigator, described at www.esaote.com, attempt to provide a user with co-registered pre-scan data. However, because in such systems the display of ultrasound is restricted to the plane of acquisition, the pre-scan data is provided as 2D slices that match the plane of the ultrasound slice, and the ultrasound and corresponding pre-operative scan cut are simply placed side-by-side for comparison, a user does not gain a 3D sense of where the ultrasound slice fits in vis-a-vis the patient space as a whole.

What is thus needed in the art is a means of correlating ultrasound scans with the 3D space and time in which they have been acquired. What is further needed is an efficient and ergonomic interface that can allow a user to easily interact with ultrasound scan data as well as pre-operative imaging and planning data in three-dimensions.

### SUMMARY OF THE INVENTION

A system and method for the imaging management of a 3D space where various substantially real-time scan images have been, or are being, acquired are presented. In exemplary embodiments of the present invention, a user can visualize images of a portion of a body or object obtained from a substantially real-time scanner not just as 2D images, but as positionally and orientationally identified slices within the relevant 3D space. In exemplary embodiments of the present invention, a user can convert such slices into volumes as desired, and can process the images or volumes using known image processing and/or volume rendering techniques. Alternatively, a user can acquire ultrasound images in 3D using the techniques of UltraSonar or 4D Ultrasound. In exemplary embodiments of the present invention, a user can manage various substantially real-time images that have been obtained, either as slices or volumes, and can control their visualization, processing and display, as well as their registration and fusion with other images, volumes or virtual objects obtained or derived from prior scans of the area or object of interest using various modalities.

### BRIEF DESCRIPTION OF THE DRAWINGS

FIG. 1 depicts a user controlling an exemplary ultrasound session with an exemplary pen and tablet two-dimensional interface according to an exemplary embodiment of the present invention;

FIG. 2 depicts a user performing three-dimensional interactions in a virtual patient space displayed stereoscopically using an exemplary three-dimensional interface according to an exemplary embodiment of the present invention;

FIG. 3 depicts a user interacting with the three-dimensional virtual patient space of FIG. 2, using a monoscopic interface according to an exemplary embodiment of the present invention;

FIG. 4 depicts an exemplary illustrative scenario where three 3D ultrasound volumes are fused with three pre-operative segmentations in an exemplary composite view according to an exemplary embodiment of the present invention;

FIG. 5 depicts exemplary user manipulations of the pre-operative segmentations and volume scans of FIG. 4 according to an exemplary embodiment of the present invention;

FIGS. 6A-6C depict exemplary preparations for a tumor removal procedure according to an exemplary embodiment of the present invention;

FIG. 7 depicts an exemplary integrated system implementing an exemplary embodiment of the present invention;

FIG. 8 depicts an exemplary external add-on system implementing an exemplary embodiment of the present invention;

FIGS. 9(a)-9(d) depict various exemplary pre-operative scenarios according to an exemplary embodiment of the present invention;

FIG. 9(e) depict an intra-operative scenario according to an exemplary embodiment of the present invention;

FIG. 9(f) depict an alternative exemplary pre-operative scenario according to an exemplary embodiment of the present invention;.

FIGS. 9(g)-9(i) respectively depict alternative exemplary intra-operative scenarios according to an exemplary embodiment of the present invention;

FIG. 10 depicts an exemplary system setup according to an exemplary embodiment of the present invention;

FIG. 11(a) depicts acquiring and storing a plurality of 2D ultrasound slices according to an exemplary embodiment of the present invention;

FIG. 11(b) depicts segmenting and blending the 2D ultrasound slices of FIG. 11(a) to produce a 3D effect according to an exemplary embodiment of the present invention;

FIG. 12 depicts scanned regions created in a virtual space according to an exemplary embodiment of the present invention;

FIG. 13 depicts an exemplary phantom used to illustrate an exemplary embodiment of the present invention;

FIG. 14 respectively depict an UltraSonar image, a reconstructed volumetric image, and a smoothed zoomed in and cropped volumetric image of the exemplary phantom of FIG. 13 according to an exemplary embodiment of the present invention;

FIG. 15 depict space tracking of two liver scans according to an exemplary embodiment of the present invention; and

FIG. 16 depicts an exemplary fusion of an ultrasound image in a single-plane with pre-operative CT data according to an exemplary embodiment of the present invention.

It is noted that the patent or application file contains at least one drawing executed in color. Copies of this patent or patent application publication with color drawings will be provided by the U.S. Patent Office upon request and payment of the necessary fee.

### DETAILED DESCRIPTION OF THE INVENTION

This present invention is directed to a system and method for the management of a 3D space where substantially real-time images have been, or are being, acquired. For purposes of illustration, exemplary embodiments of the invention will be described with reference to ultrasound images, it being understood that any equivalent substantially real-time imaging modality can be used.

In exemplary embodiments of the present invention a clinician can visualize images obtained from an ultrasound scanner not just as 2D images but as 2D slices within a particular 3D space (or alternatively as volumes within such 3D space), each acquired at a known time, and can convert such 2D slices into volumes whenever needed. In exemplary embodiments of the present invention, the method allows a user to manage the different images obtained (either as slices or volumes), and to manipulate them as well as control various display parameters, for example, their visualization (including stereoscopically), registration and segmentation.

Moreover, in exemplary embodiments of the present invention, a system can record for each acquired real-time image its 3D time and position. Therefore, in such exemplary embodiments, not only can a current image slice be displayed in its correct 3D position, but because the time of acquisition is available for each image, such methods also allow for the display of any previously acquired information at the given position. This allows for the visualization of time-variant processes, such as, for example, an injection of a contrast agent. For example, a contrast agent may be needed in order to characterize a particular lesion in liver tissue that may not be visible without it. During the time that the contrast agent is available in the relevant tissues, a system can record both the 3D position and the time of acquisition for each image. Later, for example, when a procedure is desired to be performed on the relevant tissue, such as, for example, a thermoablation, the recording of the tissue with the contrast agent flowing through it can be replayed (being co-registered to the ablation needle which can also be displayed in the 3D space, either within a current ultrasound slice, or by tracking the needle) to again visualize the lesion now no longer visible.

Thus, in exemplary embodiments of the present invention, a user can manage the entire 3D space within which ultrasound scans from a particular scanning session are obtained in a way that leads to better diagnosis and/or intervention. It is noted that the disclosed method works without “co-location” of the ultrasound images with a real patient. The fusion in exemplary embodiments is between various images, as opposed to being between a virtual world and a real patient space such as is done in certain conventional augmented reality techniques.

In exemplary embodiments of the present invention a 3D interactive system is provided that can work with either ultrasound planes (shown in their respective 3D context), volumetric reconstructions of such ultrasound information, pre-operative imaging and planning data (e.g., CT, MRI, planning pathways and selected objects in 3D data set, etc.) as well as other elements that can contribute to the procedure. This adds the ability to re-position ultrasound planes and other elements, such as an RF probe, more easily since the user can see a 3D space with “floating” objects and he can then, for example, simply move the needle or ultrasound probe to the 3D point where the floating object is perceived. This is in contrast to conventional systems, which neither provide an unrestricted display of an ultrasound (or other substantially real-time scan) plane in the context of co-registered pre-scan data, nor allow a user to freely move within the 3D space in which the real-time scan is acquired. Thus in exemplary embodiments of the present invention the facility is provided to make full use of data from prior scans such as, for example, CT or other ultrasound imaging scans, of the same patient area in an integrated manner with the substantially real-time images.

In exemplary embodiments of the present invention the coordinate positions of prior scan and real-time scans can be co-registered, allowing a user to interactively visualize the co-registered information in a way that is intuitive and precise. In so doing, acquired data can, for example, then be used to navigate a procedure, or later review a case. Such post procedural review is easily available because the 3D positions of the ultrasound planes are stored and can be analyzed after the ultrasound exploration.

The disclosed method operates via registration of ultrasound images with a virtual patient—i.e., by registering pre-operative images and or segmentations therefrom with recently acquired ultrasound data of a given patient. Alternatively, the disclosed method can operate by registering one set of ultrasound data with one or more other sets of ultrasound data, either taken at different 3D positions, or at different times, or both. In either case, in exemplary embodiments of the present invention, once various images are co-registered, fused images incorporating all or parts of the various co-registered images, as may be decided dynamically by a user, can be interactively viewed and manipulated. Thus, for example, a user can perform, use or implement any of the techniques described in any of the pending patent applications incorporated by reference above while performing an ultrasound session or ultrasound guided procedure. For example, a user can resegment and adjust any display parameters for any pre-scan data relevant to the current focus of the ultrasound imaging. Vessels form an earlier CT scan can be cropped, segmented, assigned different color look-up table values, thresholded, etc. so as to focus the current—or recent—area of interest in the ultrasound procedure. Alternatively pre-procedural planning notes, highlights and/or pathways can be dynamically and interactively brought up, hidden, or made more or less transparent as may be desired throughout the ultrasound session.

In exemplary embodiments of the present invention, the disclosed method can be integrated with the following technologies: (a) visualization of 2D ultrasound slices into a volume without the need for volume resampling (and the concomitant resampling errors), as described more fully in “UltraSonar”; and (b) a virtual interface to substantially real-time scanning machines, as described more fully in “Virtual Interface.”

Thus, in exemplary embodiments of the present invention, a special virtual interface can be used to control an interactive ultrasound scanning session. Additionally, ultrasound probes and instruments can, for example, be tracked by a 3D tracking system so that the each of the probes' and instruments' respective 3D positions and orientations can be known at all times during the ultrasound scan.

Moreover, as noted, ultrasound scanning can, for example, be preceded by pre-operative CT or MR imaging in which, for example, a segmentation of various objects or a “signature” of various organs or organelles (such as, for example, the vascular system of a liver or kidney) can be extracted to identify geometrical and topological components that can define the anatomy and pathology of the specific patient under treatment. Such a characteristic can be subsequently utilized to maintain registration between pre-operative data and real-time ultrasound scanning images or volumes.

Also, during ultrasound scanning, acquired images can, for example, be visualized using the techniques described in UltraSonar. This technique, by allowing the display of a certain number of past ultrasound slices to only slowly fade away, can allow a user to visualize 2D ultrasound slices as “pseudo-volumes” without the need for time-consuming re-sampling into actual 3D volumes and subsequent volume rendering.

Control and Display Interfaces

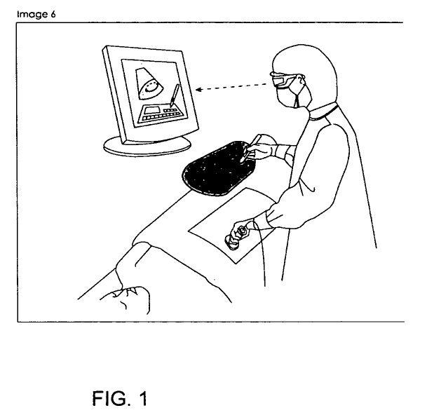

In exemplary embodiments according to the present invention a pen-and-tablet interface can be used for 2D control, as depicted in FIG. 1. With reference thereto, a user 100 can, for example, physically manipulate a pen 110 and table 120, and can thus interact with a virtual keyboard as shown at the bottom of display 130, in similar fashion as described in Virtual Interface or in A Display Apparatus. Thus, control commands such as, for example, pushing or selecting menu bars, typing in text, selecting between menu options, etc. can be mapped from the displayed virtual keyboard to 2D manipulations of the pen and tablet. The pen and table can utilize a 2D tracking device for this purpose.

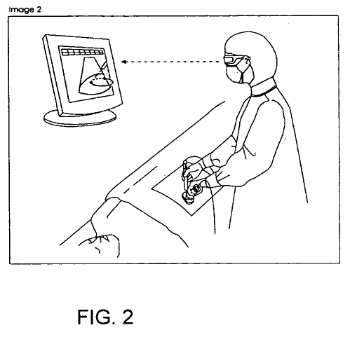

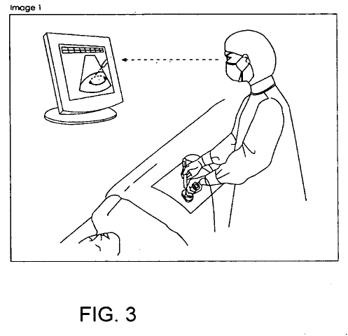

For 3D control, a 3D interface can be used as depicted in FIG. 2. With reference thereto, in exemplary embodiments of the present invention the entire interface can utilize a stereoscopic display 230 (note how the depicted scan jumps out of the screen, simulating the stereoscopic effect) inasmuch as this can afford superior depth perception, which is the key to any 3D interface. However, in alternate exemplary embodiments of the present invention the method can also be operated using a standard monoscopic interface 330, as shown in FIG. 3, thus allowing more or less standard equipment to be used in, for example, more economic or retrofit implementations of exemplary embodiments of the present invention.

3D Manipulations in 3D space

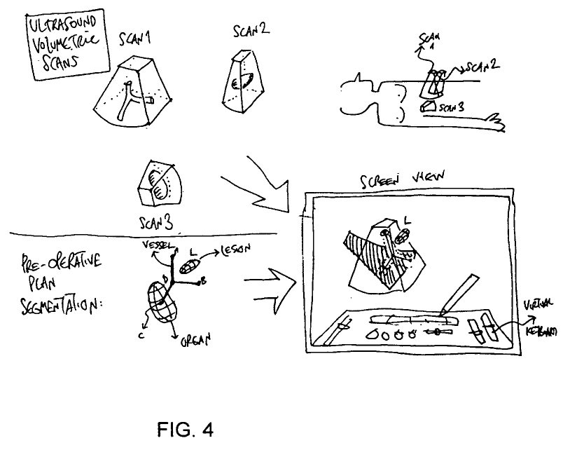

In exemplary embodiments according to the present invention, greater control and integrated imaging and display management of a 3D space where substantially real-time imaging is performed can be enabled. For purposes of illustration, in what follows an exemplary ultrasound scanning of a liver with a lesion (tumor) will be described. In the following description, it is assumed, for example, that a patient has had a pre-operative CT scan of his liver, and during a subsequent surgical planning session, three “objects” were identified by the clinician, as depicted in FIG. 4. These objects are (i) a vessel defined by three terminal points (A, B, C) and a central “hub” (point D), all connected together; (ii) a lesion L; and (iii) an adjacent organ O, for example a kidney, that serves as an anatomical landmark.

These three objects can, for example, be defined geometrically in a segmentation process and can thus be represented by polylines, polygonal meshes, and/or other graphical representations.

Given this exemplary pre-scan history, in an ultrasound scanning session a clinician can, for example, perform three corresponding volumetric ultrasound scans using, for example, an ultrasound probe with a 3D tracker. This process is illustrated in the upper right quadrant of FIG. 4. These scans can be, for example, with reference to FIG. 4, Scan 1 of blood vessel ABCD (obtained at time T₁, when a contrast medium is flowing through it, for example, at the arterial phase); Scan 2 of a lesion (obtained at time T₂, when the contrast medium has filled the liver and the lesion shows more signal (i.e., the liver is full of contrast and thus the lesion echoes back stronger to the ultrasound probe), for example, in the portal phase); and Scan 3 of the organ (obtained at yet another time T₃, at a basal phase, and at a different angle from the other two scans). Such an organ could be, for example, a kidney that can be seen without contrast. These scans can then be stored for subsequent manipulations.

Alternatively, a user could scan a given area multiple times using different ultrasound probes, where each has different acquisitional properties, and can, for example, store the scans according to the methods of the present invention. Just as in the case of using a single probe at different times with contrast, the multiple scans of the same area with the different probes will acquire different images which can then be fused to exploit the informational benefits of each probe type yet display then simultaneously in a synoptic view.

In order to fully use the information obtained from such scans, in exemplary embodiments of the present invention the pre-operative segmentations can, for example, be registered with the patient. This can be done, for example, by means of fiducial markers placed on the skin of the patient, or by any other known means. Once this is done, the three stored volumetric scans as well as the pre-operative data or any processed versions thereof (e.g., by colorizations, segmentations, constructions of mesh surfaces, etc.) are objects that a clinician or other user can manipulate in the displayed 3D space to complete a diagnosis or guide an intervention, such as, for example, insertion of a thermoablation needle. Once the three scans have been obtained, a clinician can put the ultrasound probe in its dock, and can concentrate using a pen as described above (with whatever hand he feels more dexterous) or other 3D interface to manipulate these 3D objects.

For example, a clinician can display them all at once, as is depicted, for example, in the composite view of the bottom right quadrant of FIG. 4, so that he can see the vessel from the arterial phase fused with the lesion from the portal phase, with the organ from the basal phase also visible to provide a recognizable reference.

Ergonomic Interaction

Additionally, in exemplary embodiments of the present invention, one or more switches, or other manual actuators, can be provided on or for a handheld probe to enhance 3D interactions. Because a user is generally always holding the ultrasound (or other substantially real-time image acquisition) probe, it is ergonomically convenient to allow him to control display parameters by actuating one or more buttons on the probe. For example, a button can be used to indicate when to use the probe to scan real-time or when to use it to rotate the entire virtual scene, which is a common a 3D data set interactive visualization operation. Or, more generally, in exemplary embodiments of the present invention, functionalities can be mapped to a plurality of actuators on the probe or on a footswitch, or both, that can free a user from having to continually move form the scanning area and interact with a separate interface on or within the ultrasound machine or the display (such as, for example, as is described in the Virtual Interface application).

Additionally, in exemplary embodiments of the present invention, a user can hold two devices, one per hand, where each has one or more buttons. For illustrative purposes, the exemplary case of one button is described herein. One hand can hold the acquisition device (an ultrasound probe, for example) and the other can hold any other tracked tool or probe, for example, one shaped as a pointer. With this simple arrangement, many interactions are possible with the 3D objects. For example, an ultrasound hand-held probe can operate in two modes of interaction, one in scanning mode (with button switch ON, for example) as in most ultrasound scanners, and the other in interactive mode (with the button switch in the alternate position, here OFF). The user can scan the patient by pressing the ON button on the ultrasound probe and moving the probe over the region of interest. Then, the user can release the button on the probe, and use the tracking information in the ultrasound probe to rotate the entire scene (effectively changing the viewpoint of the user over the entire 3D scene). With a second handheld tracker (say in the shape of a stylus), for example, a user can perform interactions on the individual objects in the 3D scene, for example, by reaching with the stylus into one of the previously acquired ultrasound planes, or volumes (generated either from UltraSonar, 4D Ultrasound, or a conventional volumetric ultrasound probe), and rotating them (while keeping the viewpoint of the entire scene unchanged). Alternatively, a user can reach into the RF ablation virtual probe (with the planned trajectory) and adjusting its position to a new position (for a better access after having observed structures on its path that were not visible during pre-operative planning).

In general, 3D objects in a scene (ultrasound planes, ultrasound volumes, pre-operative data like CT, segmented structures of the CT, planning pathways and information) can be assigned a bounding box around them (covering their maximum 3D extent, or a symbolic part of their extent). Additionally, for example, a bounding box can be sub-divided into different boxes, such as, for example, the corners of the box, or the edges or planes defining the box. Such a sub-part of the box can have a predefined meaning. For example, the corners of the box can be used to provide access to the object, such as, for example to rotate it, or move it to a new place, or simply to inspect it (then returning it to its original position), or to make the object invisible (while leaving a 3D marking in the position the object was to make it visible again later). Thus, the user can reach with the stylus into the desired object (say the pre-segmented tumor of the CT), and then reach into the desired operation of the object (say the corner for “inspection”), and then use the six degrees of freedom of the stylus to have a look at the tumor in all directions. Or, alternatively, for example, a user can reach into the edge of the tumor bounding box and make it invisible.

Post-Procedural Review

Once the interactive session is completed, all the information observed, as well as all of the interactive commands entered by a user, can be used for post-examination review. This is an important feature, since ultrasound revision is generally done on videos and 2D slices that move, and the 3D content is not fully appreciated, especially not as a 3D space. In such conventional pratices, one can generally save the 4D beating heart or a segment of a liver, but not the “scene” in which the different captured 2D slices and volumes were acquired. In exemplary embodiments of the present invention, in contrast, all of this material can be reviewed, looked at from different points, perhaps with a more powerful computer not practical for an operating room. The entire visual experience of the ultrasound operator can be played back, as all of the data he saw, all the pre-operative data he called up, and each command he entered can be stored. This way, ultrasound examinations can be made to be more like MR or CT studies that dissociate scanning time from diagnostics time (and are generally done by different people). Moreover, during a playback of the various views and interactive commands, all of the 3D interactive functionalities are active, so a reviewer can stop the playback, add or subtract 2D and 3D objects form the scene, and thus “second guess” the actual ultrasound session, even frame by frame, if desired, such as in forensic or mentoring contexts.

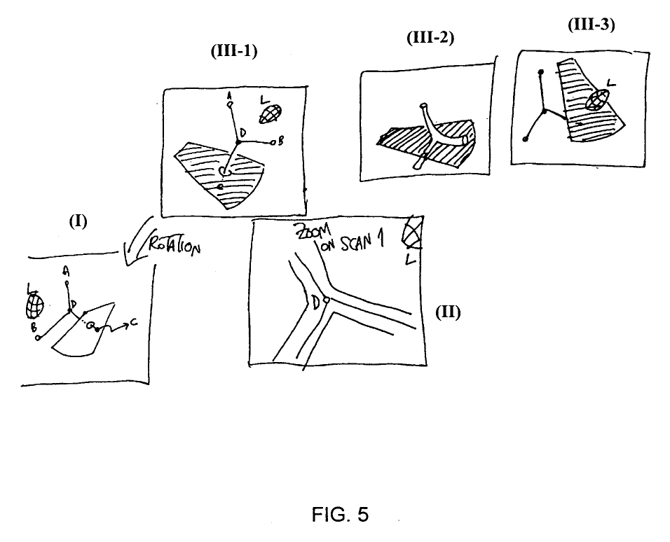

Alternatively, if the simultaneous display of all of the objects produces a confusing picture, a clinician can, for example, use a virtual interface to select which objects to view. Once such a selection is made, the clinician can, for example, use the pen or other tracked handheld tool to control the way in which to see the objects by performing various 3D volumetric operations upon them, such as, for example, described in detail in Zoom Slider, Zoom Context and 3D Matching, or as otherwise known in the art. Some of these possibilities are next described with reference to FIG. 5.

For example, with reference to view (I) of FIG. 5, objects can be rotated, thus offering a point of view that is different from the viewpoint used during the scan. This can, for example, reveal parts that were obscured from the viewpoint used during the scan. Although the viewpoint can be changed by a user during a scan, users are often too busy doing the ultrasound scan to do this. Further, zooming operations can be effected, as depicted in view (II). Here again, this can reveal detail not easily appreciated from the original scanning viewpoint. Because a viewpoint used during a scan is not necessarily the optimal one for viewing all objects, such exemplary 3D interactions post-scan can greatly enhance examination of a patient. Further, a clinician can, for example, use a pen or other handheld tool to select objects from a virtual interface, and subsequently use the pen to move objects in the 3D space near the patient so that the objects appear on the display floating above the patient (as depicted in the display, the virtual patient being the element of the composite image, as noted above).

Once a sufficiently clear picture of the anatomy and pathology is obtained, a user can, for example, position a virtual needle into the scene (for example, a line drawn in 3D space) to mark the best approach to the lesion. Such a virtual needle can then remain floating in its 3D position on the display as a reference.

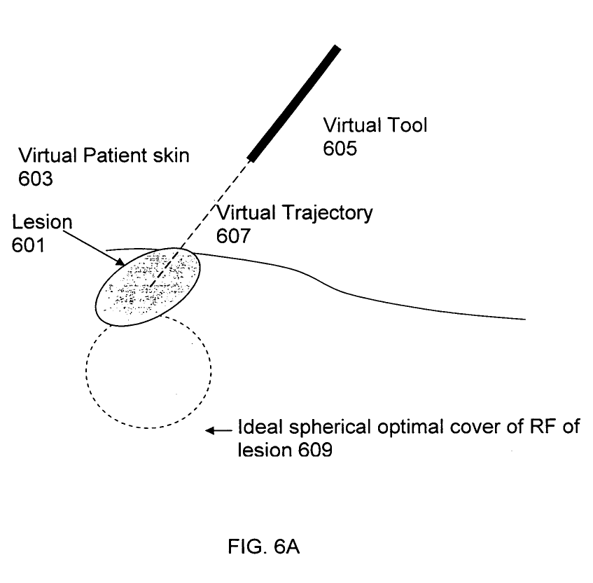

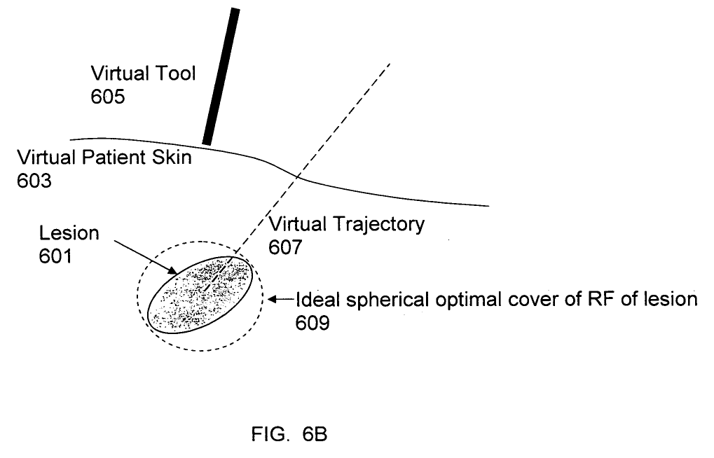

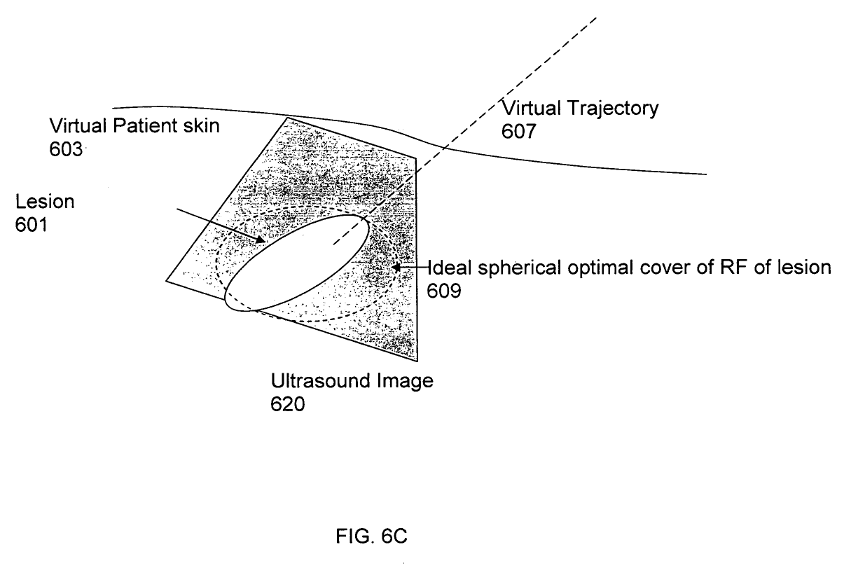

A clinician can then, for example, again activate the ultrasound probe and bring it into the 3D space of the patient, as depicted in FIGS. 6A-6C, as described below. The ultrasound probe can thus, for example, show a live image from the patient, and in exemplary embodiments of the present invention this live image can be displayed as surrounded by the objects (i.e., the volumes from earlier ultrasound scans in the current session and/or the segmentations from other pre-operative scans, as may be useful to a given user at a given time) in their correct positions and orientations relative to the current scan slice, as shown in views III-1, III-2 and III-3 of FIG. 5. With reference thereto, III-1 shows the lesion object L and the vascular signature ABCD topologically correctly fused with the current ultrasound scan slice, III-2 shows the blood vessel of Scan 1 topologically correctly fused with the current ultrasound scan slice, and III-3 shows the lesion object L and the vascular signature ABCD topologically correctly fused with the current ultrasound scan slice, where the position and orientation of the current scan slice has moved to between lesion object L and vascular characteristic ABCD, or upwards and more vertical from the position and orientation of the current scan slice as depicted in III-1. This process can thus be used to confirm the respective relative positions of the various stored objects to the patient (actually the virtual patient, there being some less than perfect correspondence between the real and virtual patients).

Finally, in the case of an intervention, a clinician can, for example, move the live ultrasound probe to the position of the virtual needle so that it can be confirmed that the lesion is within reach from that place and can proceed with the intervention in the conventional way. This process is next described.

Because, in exemplary embodiments according to the present invention, a 3D virtual patient space can be managed like any other 3D data set with a variety of co-registered objects, a user can create surgical planning data and add it to the displayed composite image. Thus, with reference to FIG. 6, a virtual tool, for example, can be used to plan the optimal direction of an ablation, as next described.

With reference to FIG. 6A, an exemplary virtual tool 605 can be, for example, moved by a user to an ideal position (i.e., here the center of a sphere which is the idealized shape utilized in this example to model a tumor) to hit a tumor 601 completely. A virtual trajectory 607 can then, for example, be projected inside a patient's virtual skin 603. With reference to FIG. 6B, the exemplary virtual tool 605 can, for example, then be moved away from the ideal tumor ablation position leaving behind an ideal path 607. With reference to FIG. 6C an exemplary ultrasound probe can, for example, then be brought back to the 3D position indicated by virtual trajectory 607, to confirm the position of the actual lesion. This generates ultrasound image 620 which displays a real-time image of the actual lesion in topological context of the virtual trajectory 607 and ideal RF envelope for ablation of the lesion, created as shown in FIGS. 6A and 6B. As described above, a user can, for example, choose and control the virtual tool and virtual trajectory creation functions, as well as the creation of a virtual ideal tumor “hit point” by interacting with the data via a virtual interface.

Exemplary System Implementation

In exemplary embodiments according to the present invention, an exemplary system can comprise, for example, the following functional components:
1. An ultrasound image acquisition system;
2. A 3D tracker; and
3. A computer system with graphics capabilities, to process an ultrasound image by combining it with the information provided by the tracker.

An exemplary system according to the present invention can take as input, for example, an analog video signal coming from an ultrasound scanner. A standard ultrasound machine generates an ultrasound image and can feed it to a separate computer which can then implement an exemplary embodiment of the present invention. A system can then, for example, produce as an output a 1024×768 VGA signal, or such other available resolution as can be desirable, which can be fed to a computer monitor for display. Alternatively, as noted below, an exemplary system can take as input a digital ultrasound signal.

Systems according to exemplary embodiments of the present invention can work either in monoscopic or stereoscopic modes, according to known techniques. In exemplary embodiments according to the present invention, stereoscopy can be utilized inasmuch as it can significantly enhance the human understanding of images generated by this technique. This is due to the fact that stereoscopy can provide a fast and unequivocal way to discriminate depth.

Integration into Commercial Ultrasound Scanners

In exemplary embodiments according to the present invention, two options can be used to integrate systems implementing an exemplary embodiment of the present invention with existing ultrasound scanners:
1. Fully integrate functionality according to the present invention within an ultrasound scanner; or
2. Use an external box.

Each of these options are next described

Full Integration Option

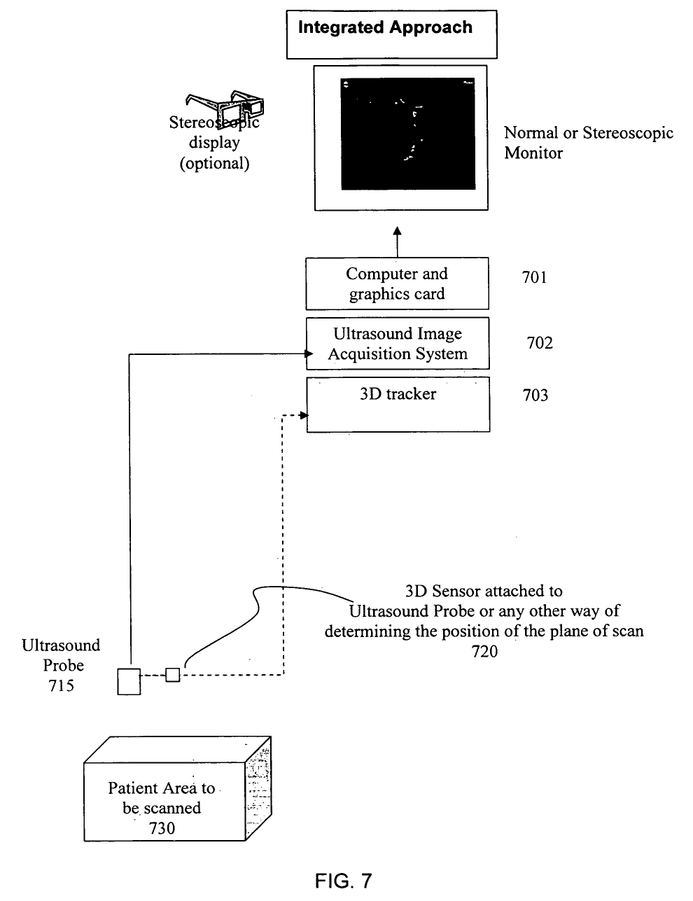

FIG. 7 illustrates an exemplary system of this type. In an exemplary fully integrated approach, ultrasound image acquisition system 701, a 3D tracker 702 and a computer with graphics card 703 can be wholly integrated. In terms of real hardware, on a scanner such as, for example, the Technos™ MPX from Esaote S.p.A. (Genoa, Italy), full integration can easily be achieved, since such a scanner already provides most of the components required, except for a graphics card that supports the real-time blending of images. Optionally, any stereoscopic display technique can be used, such as autostereoscopic displays, or anaglyphic red-green display techniques, using known techniques. A video grabber is also optional, and is in some exemplary embodiments can be undesired, since it would be best to provide as input to an exemplary system an original digital ultrasound signal. However, in other exemplary embodiments of the present invention it can be economical to use an analog signal since that is what is generally available in existing ultrasound systems. A fully integrated approach can thus take full advantage of a digital ultrasound signal. As can be seen with reference to FIG. 7, an area desired to be scanned 730 can be scanned by an ultrasound probe 710 which feeds an ultrasound signal to the ultrasound image acquisition system 702. Additionally, the 3D position of the ultrasound probe 715 can be tracked by 3D tracker 703, by, for example, 3D sensor 720 which is attached to ultrasound probe 715.

External Box Option

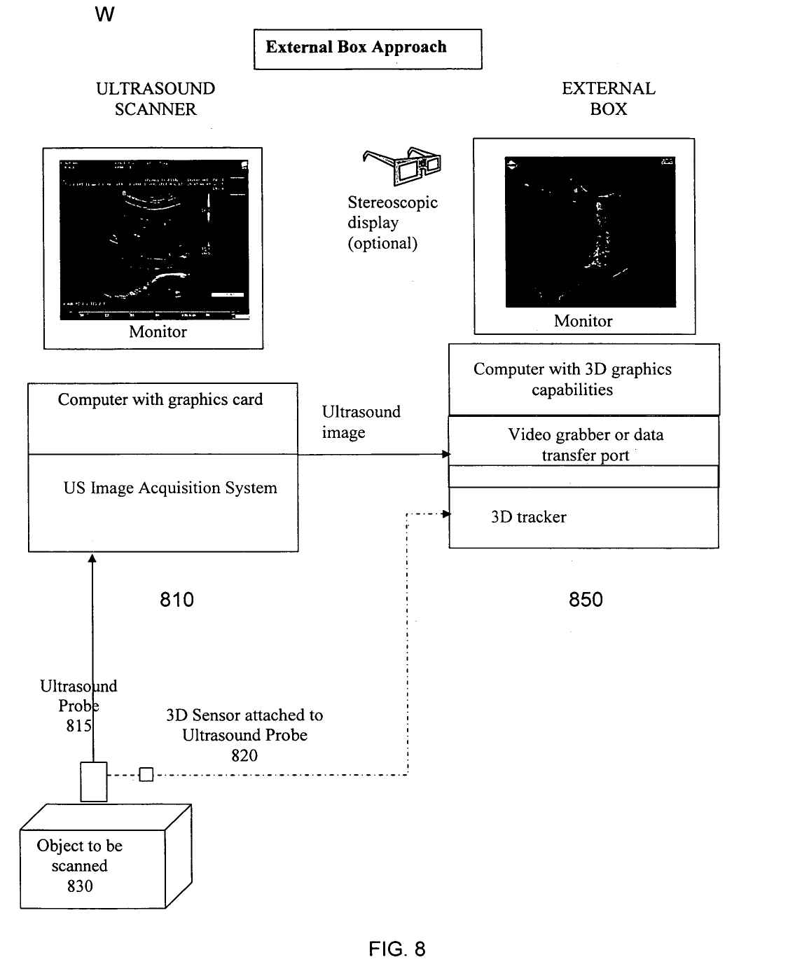

FIG. 8 illustrates an exemplary system of this type. This approach can utilize a box 850 external to the ultrasound scanner 810 that takes as an input the ultrasound image (either as a standard video signal or as a digital image), and provides as an output a 3D display. Such an external box 850 can, for example, connect through a video analog signal. As noted, this can not be an ideal solution, since scanner information such as, for example, depth, focus, etc., would have to be obtained by image processing on the text displayed in the video signal. Such processing can have to be customized for each scanner model, and can be subject to modifications in the user interface of the scanner. A better approach, for example, is to obtain this information via a data digital link, such as, for example, a USB port, or a network port. An external box 850 can be, for example, a computer with two PCI slots, one for the video grabber (or a data transfer port capable of accepting the ultrasound digital image) and another for the 3D tracker. Operationally, the same functionality of, and mutual relationships between, ultrasound probe 815, object to be scanned 830 and 3D tracker 820 as was described for corresponding elements 715, 730 and 720 with reference to FIG. 7 would apply using the external box option depicted in FIG. 8.

It is noted that in the case of an external box approach it is important that there be no interference between the way of displaying stereo and the normal clinical environment of the user: there will be a main monitor of the ultrasound scanner, and if the stereo approach uses shutter glasses, the different refresh rates of the monitor will produce visual artifacts (blinking out of sync) that can be annoying to a user. Thus, in the exemplary external box approach the present invention needs to be used with either a polarized screen (so that the user wears polarized glasses that will not interfere with the ultrasound scanner monitor; and additionally, will be lighter and will take away less light from the other parts of the environment, specially the patient). Alternatively, in exemplary embodiments of the present invention an autostereoscopic display can be utilized, so that no glasses are required.

Exemplary Fused Images

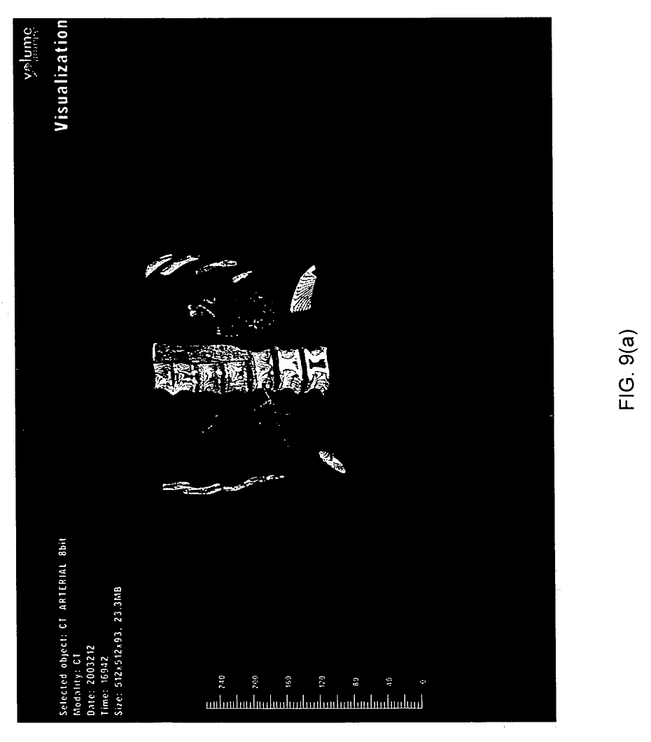

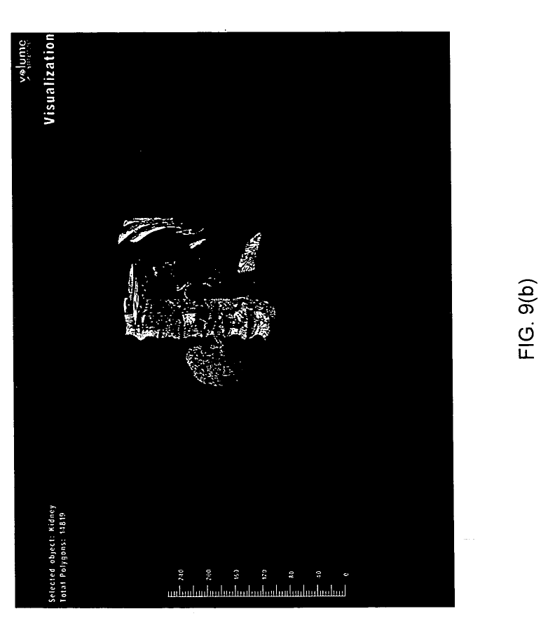

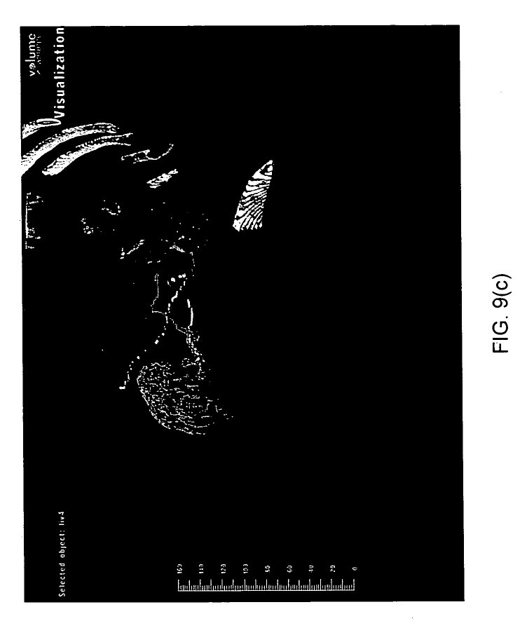

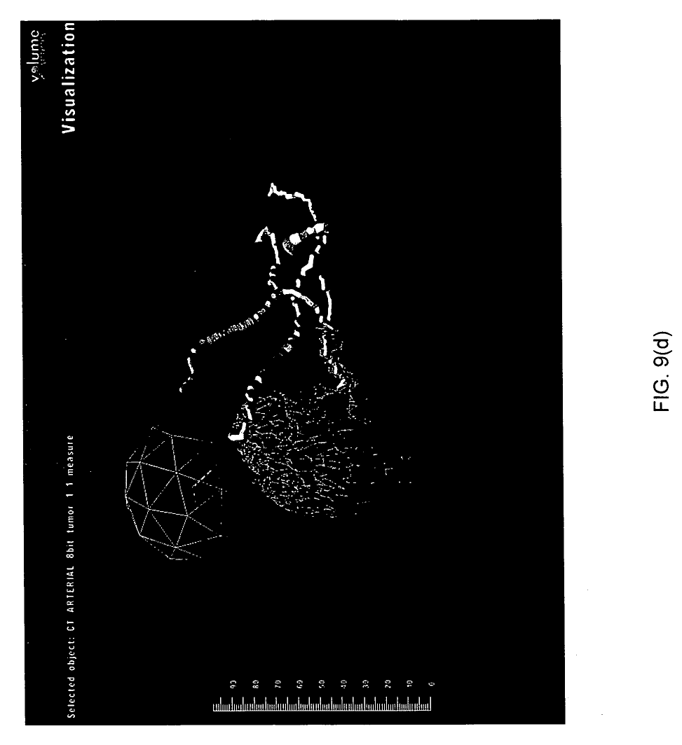

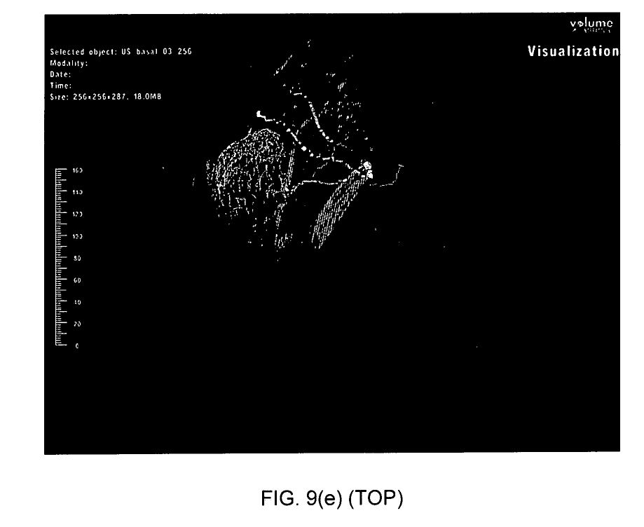

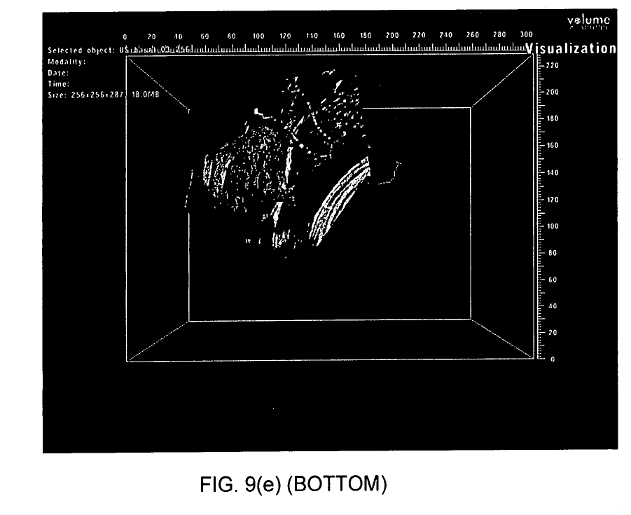

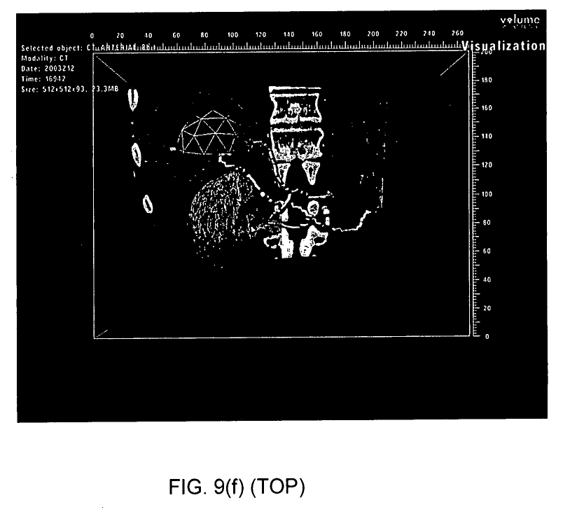

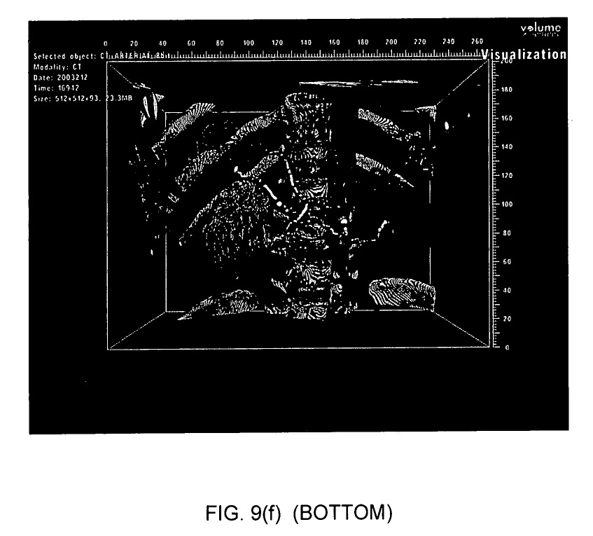

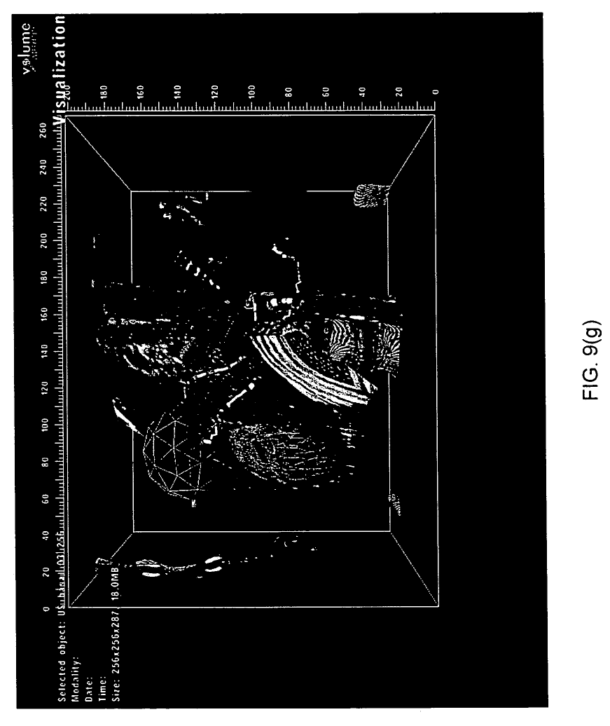

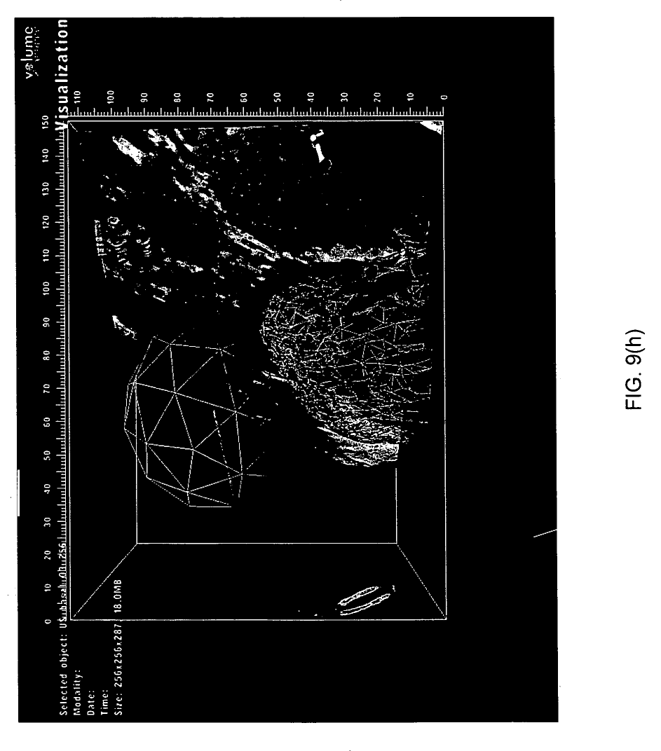

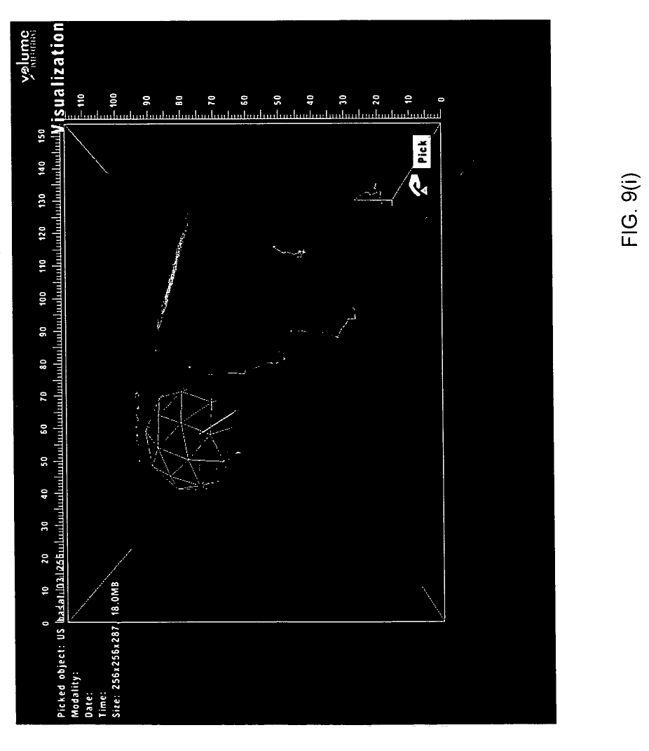

FIGS. 9(a) through 9(i) depict exemplary images that can be obtained according to an exemplary embodiment of the present invention. They depict various pre-operative data, such as, for example, CT, and virtual objects derived therefrom by processing such data, such as, for example, segmentations, colorized objects, etc. Most are simulated, created for the purposes of illustrating exemplary embodiments of the present invention. Some only depict pre-operative data (i.e., the pre-operative scenarios), while in others (i.e., the “interoperative scenarios”), such virtual objects are fused with a substantially real-time ultrasound image slice in various combinations according to exemplary embodiments of the present invention. These exemplary figures are next described in detail.

FIG. 9(a) is an exemplary pre-operative scenario of a CT of a patient with a liver and kidney tumor, displayed revealing exemplary kidneys and liver (and dorsal spine).

FIG. 9(b) is an exemplary pre-operative scenario of an anatomical “signature” extracted from CT data, with an exemplary kidney segmented as polygonal mesh. Such signatures can be used, in exemplary embodiments of the present invention, much as natural fiducials, i.e., as navigational guides to a user. (As used herein the term “signature” refers to a unique 2D or 3D structure of a given anatomical object, such as a liver's vascular system, the outline of a kidney, etc. The term “characteristic” is sometimes used for the same idea.)

FIG. 9(c) is an exemplary pre-operative scenario of exemplary arteries which were added to the exemplary signature, segmented from the aorta and shown as tubular structures in a zoomed view.

FIG. 9(d) is an exemplary pre-operative scenario of an exemplary tumor which was added to the exemplary signature, shown segmented as a polygonal mesh with three vectors indicating dimensions in the x, y, z axis.

FIG. 9(e) depict an exemplary intra-operative scenario showing an ultrasound plane fused with the exemplary signature extracted from CT data in a zoomed view. The upper image shows a semitransparent ultrasound plane that reveals the extracted vessels behind the ultrasound plane, while the lower image shows an opaque ultrasound plane. Whether an ultrasound plane appears as opaque or transparent is a display parameter that can be, in exemplary embodiments, set by a user.

FIG. 9(f) depict an exemplary pre-operative scenario showing the exemplary signature in the context of the CT data. The kidney is segmented as polygonal mesh, exemplary arteries are segmented as tubular structures and an exemplary tumor is segmented as an ellipse. The bottom image has the CT data colorized via a color look-up table.

FIG. 9(g) is an exemplary intra-operative scenario showing an exemplary “live” ultrasound image fused with the exemplary signature and pre-operative CT data.

FIG. 9(h) is an exemplary intra-operative scenario showing an exemplary zoomed view of a “live” ultrasound image fused with the exemplary signature and pre-operative CT data.

FIG. 9(i) is an exemplary intra-operative scenario showing a different angle of a zoomed view showing a segmented tumor fitting inside the dark area of a live ultrasound image (corresponding to the tumor). Also visible are exemplary CT vessels, segmented arteries, and vessels in the ultrasound image.

Thus, FIGS. 9(a) through (i) illustrate a few of the various possibilities available to a user in exemplary embodiments of the present invention. Pre-operative segmentations and virtual objects, or even 2D and/or 3D ultrasound images form moments before, can, in such exemplary embodiments, be fused with live ultrasound data due to the co-registration of the patient (i.e., the virtual patient) with the real-time ultrasound by means of the 3D tracking system. In this manner as much context as is desired can be brought in and out of the fused view and the components of that view can be interactively manipulated. Thus a user truly has control of the virtual 3D space in which he is carrying out an ultrasound imaging session.

Exemplary System

An exemplary system according to an exemplary embodiment of the present invention which was created by the inventors is described in the paper entitled “SONODEX: 3D SPACE MANAGEMENT AND VISUALIZATION OF ULTRASOUND DATA” provided in Exhibit A hereto, which has been authored by the inventors as well as others. Exhibit A is thus fully incorporated herein by this reference. The exemplary system presented in Exhibit A is an illustrative example of one system embodiment of the present invention and is not intended to limit the scope of the invention in any way.

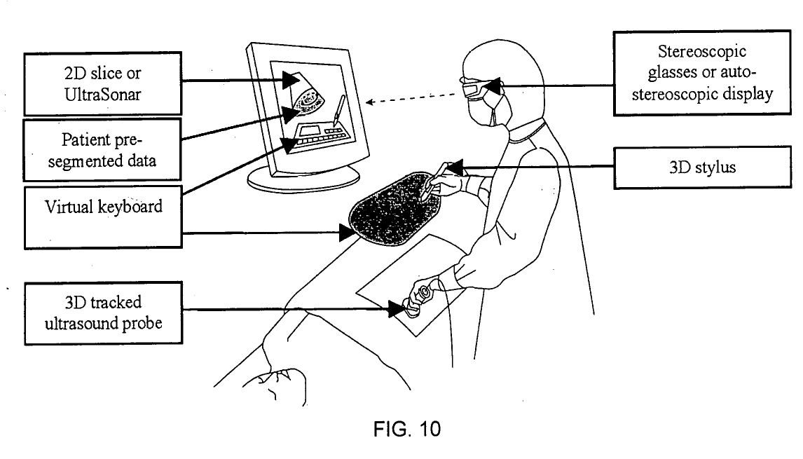

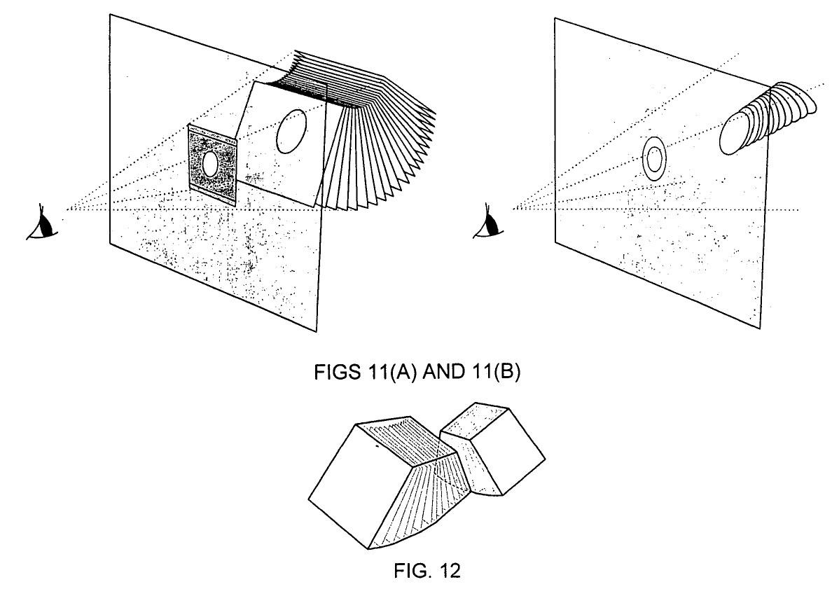

With reference to Exhibit A, FIGS. 10-16 correspond to FIGS. 1-7 of the article provided in Exhibit A. Thus, FIG. 10 (FIG. 1 in Exhibit A) depicts an exemplary system setup according to an exemplary embodiment of the present invention. FIG. 11(a) (FIG. 2 (Left) in Exhibit A) and 11(b) (FIG. 2 (Right) in Exhibit A) depict acquiring and storing a plurality of 2D ultrasound slices and segmenting and blending the 2D ultrasound slices to produce a 3D effect, respectively, using the UltraSonar technique. FIG. 12 (FIG. 3 in Exhibit A) depicts scanned regions created in an exemplary virtual space by recording ultrasound images in time and 3D space as described above.

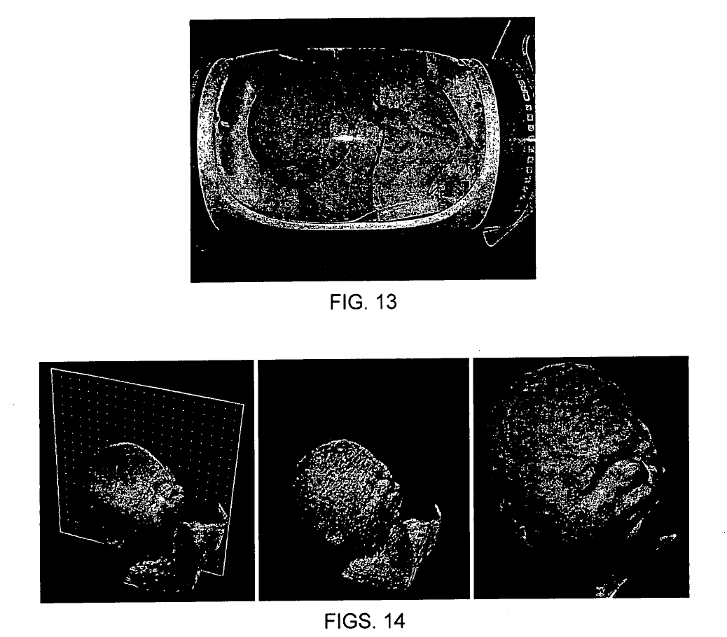

FIG. 13 (FIG. 4 in Exhibit A) depicts an exemplary phantom used to illustrate the functionalities of the exemplary embodiment of the present invention described in Exhibit A, and FIG. 14 (FIG. 5 in Exhibit A) respectively depict an UltraSonar image (FIG. 5 (Left) in Exhibit A), a reconstructed volumetric image (FIG. 5 (Center) in Exhibit A), and a smoothed, zoomed in and cropped volumetric image (FIG. 5 (Right) in Exhibit A) of the exemplary phantom.

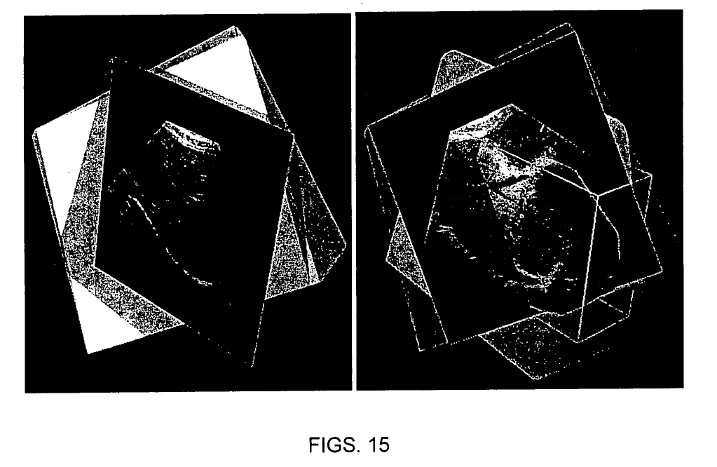

FIG. 15 (FIG. 6 in Exhibit A) depict space tracking of two liver scans (Left) according to an exemplary embodiment of the present invention, where one scan is reconstructed into a volume and the other scan is superimposed in single-slice mode in the same space (Right).

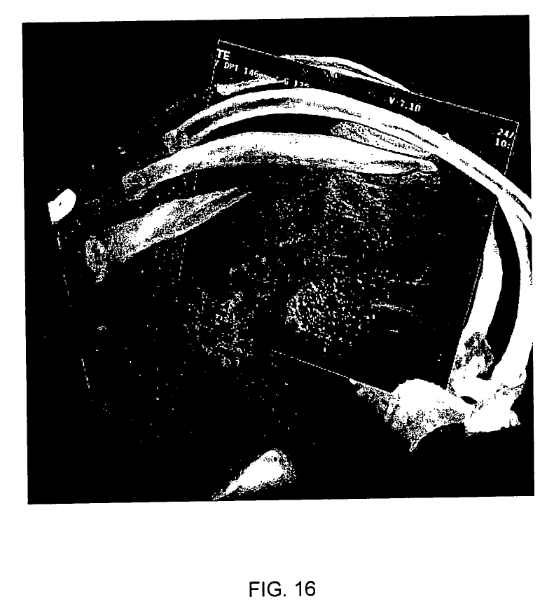

Finally, FIG. 16 (FIG. 7 in Exhibit A) depicts an exemplary fusion of an ultrasound image in a single-plane with pre-operative CT data according to an exemplary embodiment of the present invention.

While the present invention has been described with reference to certain exemplary embodiments, it will be understood by those skilled in the art that various changes may be made and equivalents may be substituted without departing from the scope of the invention. For example, the system and methods of the present invention can apply to any substantially real-time image acquisition system, not being restricted to ultrasound, and to fuse with such substantially real-time imaging system previously acquired or created data of any type, including, for example, enhanced volumetric data sets created form various imaging, contouring or other data sources. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed:

1. A method of managing a 3D space in which substantially real-time images are acquired, comprising:
acquiring substantially real-time images of an object or body;
co-registering prior image data to the 3D space from which the substantially real-time images were acquired;
tracking a scan probe and a handheld tool in 3D;
using the tracking information from the scan probe to fuse images from or derived from prior scans of the object or body to one or more substantially real-time images of the object or body; and
using the tracking information from the handheld tool to control display parameters and manipulational operations on the one or more substantially real-time images.

2. The method of claim 1, wherein the substantially real-time images include 4D scans.

3. The method of claim 2, wherein one or more of the 4D scans are acquired using a contrast agent that enhances different portions of the object or body at different times.

4. The method of claim 2, wherein one or more of the 4D scans are acquired using different scan probes each having different imaging properties.

5. The method of claim 1, wherein the prior scans include 2D or 3D images of the same modality as the substantially real-time images.

6. The method of claim 1, wherein the prior scans include images from different modalities than the substantially real-time images.

7. The method of claim 1, wherein the prior scans include images and/or virtual objects derived from processing images or scans from different modalities than the substantially real-time images.

8. A system for managing the 3D space in which substantially real-time images are acquired, comprising:
a substantially real-time image acquisition system with a scan probe;
a 3D tracker; and
a computer system with graphics capabilities,
wherein the computer system processes one or more acquired ultrasound images by using information provided by the tracker.

9. The system of claim 8, wherein the substantially real-time image acquisition system is an ultrasound machine.

10. The system of claim 8, wherein said processing includes one or more of (i) co-registering prior image data to the 3D space from which the substantially real-time images were acquired, (ii) using tracking information from the scan probe to fuse images from or derived from prior scans of the object or body to one or more substantially real-time images of the object or body; and (iii) using tracking information from a handheld tool to control display parameters and manipulational operations on the one or more substantially real-time images.

11. A method of ablating one or more tumors, comprising:
acquiring a tracked 4D scan of an area of a body using a contrast agent;
tracking a scan probe and a handheld ablation tool in 3D;
using the tracking information from the probe to fuse prior scans of the object or body, or images derived therefrom, to one or more substantially real-time images of the area of the body; and
using the tracking information from the handheld ablation tool to plot a virtual path to each tumor prior to insertion.

12. The method of claim 11, wherein one of said images derived form a prior scan includes a segmentation of a tumor.

13. The method of claim 11, further comprising using the tracking information from the probe to create and fuse surgical plan data with the one or more substantially real-time images of the area of the body.

14. The method of claim 11, further comprising acquiring one or more tracked 4D scans of an area of a body using multiple ultrasound probes each having different imaging properties.

15. A 3D space management system for ultrasound imaging, comprising:
a stereoscopic display;
a data processor with memory;
a 3D tracked ultrasound probe; and
a 3D interaction tool or mouse,
wherein a user controls the ultrasound probe with one hand and manipulates images with the other;

16. The system of claim 15, wherein the images are either acquired ultrasound images or images generated by the data processor from previously stored scan data.

17. The system of claim 16, wherein the acquired ultrasound images are either 2D or 3D and are stored with a time stamp, size, orientation, position and color look-up table.

18. The system of claim 17, wherein 3D ultrasound images are acquired using one of UltraSonar or 4D Ultrasound techniques.

19. The system of claim 18, wherein an entire organ can be reconstructed as a virtual object by combining multiple saved 3D ultrasound images.

20. The system of claim 16 wherein acquired ultrasound images of various types are displayed with virtual images or volumes from prior scan data to interactively display multiple aspects of a region or object of interest.
